(19) United States
(12) Patent Application Publication
(10) Pub. No.: US 2023/0074949 A1
(43) Pub. Date: Mar. 9, 2023

(54) REMOTE SENSING OF PHYSIOLOGICAL DYNAMIC ELECTROMAGNETIC BIG DATA

(71) Applicant: Fenghua LIU, Guiyang, Guizhou (CN)

(72) Inventor: Fenghua LIU, Guiyang, Guizhou (CN)

(21) Appl. No.: 17/767,865

(22) PCT Filed: Oct. 9, 2020
(86) PCT No.: PCT/CN2020/000236

§ 371 Date: Nov. 18, 2022

(30) Foreign Application Priority Data
Oct. 9, 2019 (CN) 201910953194.X

(51) Int. Cl. G01N 21/25; A61B 5/18; A61B 5/16; A61B 5/00; A61B 5/0507; G01N 21/47

(52) U.S. Cl. CPC G01N 21/25; A61B 5/18; A61B 5/165; A61B 5/4845; A61B 5/0507; G01N 21/47; A61B 2503/40; A61B 2503/22

(57) ABSTRACT

The title of the invention is the remote sensing of physiological dynamics electromagnetic big data, the related technology belongs to the field of remote sensing of physiology or psychology. The technical problem to be solved by the present invention is the remote sensing of physiological or psychological electromagnetic dynamics of human, animal or plant individuals, the related electromagnetic media could be nucleon, electron, atom, molecule, ion, water, fluid, blood, DNA, RNA, amino acid, sugar, fat, protein, cell, tissue, organ, bone, fruit, flower, leave, stem, bark, and root, etc., the 30 μm-1000 million meters band electromagnetic waves could be used to extract the reflection spectra of partial or entire human, animal or plant individual electromagnetic media in different physiology or psychology states, single or multiple sensitive wavelengths or wavebands of spectrum troughs could be chosen as the electromagnetic wave probes, which could be used to irradiate partial or entire human, animal or plant individual electromagnetic media and extract the reflection waves, the electromagnetic dynamics of the human, animal or plant individuals in different physiology or psychology states could be transformed to the different electromagnetic indexes, such as the frequency, reflection rate and phase values, the major applications of the invention might be the drunk driving, fatigue driving, road rage driving, big data and big ecology, etc.

### TECHNICAL FIELD

The technical scheme claimed for protection belongs to the field of remote sensing of physiology or psychology.

### BACKGROUND

When human, animal and plant are irradiated with the visible light, the reflection, scattering, refraction, absorption, transmission and diffraction could happen. The reflection-scattering waves of visible light are generally used to extract the color features of biological surfaces. The reflection-scattering data could represent the surface colors of biological individuals qualitatively, semi-quantitatively and quantitatively. Therefore, The reflection-scattering waves of visible light could realize the remote sensing of biological surface colors.

### SUMMARY

The technical problem to be solved by the present invention is the remote sensing of the physiological or psychological dynamics of human, animal and plant individuals at near light speed regardless of the rain-snow-fog weathers. The invention can be used in various aspects including drunk driving, fatigue driving, big data, and big ecology, etc. The physiological media such as nucleon, electron, atom, molecule, ion, water, fluid, blood, DNA, RNA, amino acid, sugar, fat, protein, cell, tissue, organ, bone, fruit, flower, leave, stem, bark and root, etc. could construct the biological individuals from the microscopic level to the macroscopic level, while essentially demonstrating the nucleon-electron clusters electromagnetic nature of the biological individuals. The physiological or psychological dynamics related to the biochemical physics are essentially the electromagnetic fluctuations. The stimuli to eye, ear, nose, tongue, body and mind by internal psychological factors such as joy, anger, worry, anxiety, sadness, fear and scare, or external environmental factors would cause electromagnetic changes of human, animal or plant individuals. The terahertz waves, millimeter waves, centimeter waves, decimeter waves, meter waves, radio frequency waves and low-frequency waves in the 30 μm-1000 million meters band could interact with the biological nucleon-electron clusters at the intrinsic frequencies or frequency bands. The penetration depth, action medium and action nature of the electromagnetic waves of different wavelengths at the biological interface could change accordingly with the wavelengths. The reflection, scattering, refraction, absorption, transmission or diffraction waves obtained by irradiating human, animal or plant individuals with single or multiple wavelengths or wavebands in the 30 μm-1000 million meters band could characterize the strong-weak electromagnetic properties of the physiological or psychological media such as nucleon, electron, atom, molecule, ion, water, fluid, blood, DNA, RNA, amino acid, sugar, fat, protein, cell, tissue, organ, bone, fruit, flower, leave, stem, bark and root, etc. Generally, the reflection-scattering wave data could be used to characterize the biological electromagnetism of human, animal and plant individuals in different physiological or psychological states, the comparison of the frequency, absorption or phase data of different physiological or psychological states could be used to realize the remote sensing of the physiological or psychological dynamics of the biological individuals. The reflection-scattering wave spectra of living organism individuals could be the dynamic data, some troughs could be relatively stable, some troughs show greater variability, some new troughs might show up. To the high-concern targets such as drivers, the electromagnetic data can be updated in a short period of time for the remote sensing of alcohol, fatigue or anger of the drivers in order to better avoid the accidents or other losses, in the meantime, a longer period of time for data update can be set for the general-concern targets. Because of the limited power density and very short action time on human, animal and plant individuals, the irradiation effects could be ignorable, the present invention is safe technically. The penetration depth of the 0.35 μm-0.75 μm band visible light is smaller than 0.75 μm at the biological interface theoretically. Limited by the property of visible light waveband, the physiological information extracted by visible light is the surface data of generally opaque organisms. Meanwhile, the qualitative ability of visible light is mainly manifested in color identification, while the superficial color data could show very limited physiology application values. The rain, snow and fog could show serious interferences to the visible light technology due to the wavelength range. The plane angle and elevation angle of incident visible light are limited while extracting the characteristic face information relevant to psychological dynamics, the characteristic psychology information could not be collected when the plane angle and the elevation angle are beyond certain values. In contrast, the 30 μm-1000 million meters band electromagnetic waves could extract three-dimensional exterior-interior electromagnetic information of the biological individuals. While the visible light interface detection could only extract the color data of red, orange, yellow, green, cyan, blue and violet at the sub-micrometer depth, the 30 μm-1000 million meters band electromagnetic waves could extract the exterior-interior electromagnetic data of non-visible light property at various depths ranging from the micrometer level to the one thousand million meters level theoretically. Therefore, the data volume extracted by the 30 μm-1000 million meters band could significantly exceed the data volume collected by the visible light, meanwhile, the different wavelengths lead to the different electromagnetism properties of the data thereof. The 30 μm-1000 million meters band electromagnetic waves with longer wavelengths and better ability against the rain-snow-fog interferences could extract electromagnetic information of human, animal and plant individuals at various observation angles and realize the remote sensing of physiological or psychological dynamics regardless of day, night, rain, snow and fog. Therefore, the present invention is the significantly advanced technology while comparing with the closest current visible light photography technology.

### DETAILED DESCRIPTION

Although human, animal and plant individuals are diverse in their outlooks, essentially they are the different electromagnetic structures comprising of astronomical nucleon-electron clusters with different properties, quantities and distributions. All of the biological physiology, psychology and external environmental influences could be essentially manifested as electromagnetic fluctuations, the 30 μm-1000 million meters band electromagnetic waves could be used to irradiate human, animal and plant individuals and the relevant electromagnetic information could be extracted from the reflection-scattering waves, according to the mechanisms of Rayleigh scattering, Raman scattering and Mie scattering the scattering directions could be distributed in the front, back or other directions, theoretically the scattering wave could be detected at the reflection wave detection point, therefore, the scattering wave at the reflection direction could be combined to the reflection wave and detected as the overall reflection wave in the actual application. The applications of UV, X-ray or gamma ray with higher frequency might be restricted due to the higher energy which might hurt the organisms. The biological electromagnetic dynamics corresponding to the physiology, psychology or external influences could be classified as the continuous fluctuations and the significant differences. The physiological electromagnetic dynamics corresponding to wakefulness, sleep and exercise could be significantly different from each other. The psychological electromagnetic dynamics of joy, anger, worry, anxiety, sadness, fear and scare could be significantly different from the calm mood state. The environmental factors such as day, night, drought, high temperature or snow might lead to significant changes in the physiological electromagnetic dynamics of plant individuals.

The physiological or psychological states of human, animal and plant individuals could be determined qualitatively and quantitatively by measuring the small or significant electromagnetic changes of their nucleon-electron clusters. The standard electromagnetic parameters, which includes the frequency, wavelength, incident light intensity, polarization, plane angle, elevation angle, distance, pulse width, and sampling gate width, etc., should be decided through the experiments to guarantee the correctness, repeatability, reliability and comparability of the data in the actual applications, the 30 μm-1000 million meters band electromagnetic waves could be used to extract the reflection spectrum of partial or entire human media in normal physiological state, the similar spectra of human individuals could show single or multiple reflection spectrum troughs, which could indicate sensitive wavelengths or wavebands where the specific electromagnetic waves could interact strongly with the nucleon-electron clusters at the specific frequencies or frequency bands, single or multiple sensitive wavelengths or wavebands of spectrum troughs could be chosen as the electromagnetic wave probes, which could be used to irradiate partial or entire human media and extract the reflection waves, the electromagnetic fluctuations of human nucleon-electron clusters could be transformed to single or multiple reflection rates and phases values with fluctuations, which could be the electromagnetic indexes to represent the human individual normal physiological state; the sleeping state and the exercise state could show some significantly different reflection spectrum troughs from the normal physiological state, the same procedures could be used to transform human individuals to respective sleeping indexes or exercise indexes.

The harm caused by drunk driving is well known. The alcohol amount is usually measured by the breath test or the blood test, the principle could be the detection of gas or liquid ethanol molecule CH₃CH₂OH in the gas or liquid mixture. The current methods are slow, the inspection capability could be negligible while comparing with the traffic flow, in the meantime, the detection could interfere with the normal traffic flow of non-drunk driving. The human physiological structure is essentially the mixed electromagnetic structure of gas, liquid and solid. Once the ethanol molecules enter the human body, some free ethanol molecules could spread gradually and then continue the circulation in the body, while some ethanol molecules could react with corresponding components to produce a series of biochemical products, meanwhile, the ethanol molecules and the series of relevant biochemical products could be metabolized by the physiological functions and gradually discharged from the body. There is no question that the physiological electromagnetic changes caused by ethanol molecules could be characterized by the 30 μm-1000 million meters band electromagnetic wave spectrum, the enough person number could be arranged to satisfy the statistic requirement and guarantee the correctness, repeatability, reliability and comparability of the data, under the standard experiment parameters, the reflection spectra of non-drinking driver and drunk driver could be extracted for comparison, the reflection spectrum of partial or entire drunk driver media will show some significant changes while comparing with the non-drinking physiological state spectrum, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves, in theory, the most sensitive wavelength or waveband from the newly appeared trough in the drunk-driver spectra could be chosen as the electromagnetic probe because it could be not necessary to rely on the comparison of the non-drinking state reflection data and the drunk state reflection data of the same driver and its technology application could be easily developed, then the reflection rate differences and the phase differences at the chosen frequency or frequency band could be defined as the alcohol indexes to reveal the drunk drivers, through establishing the mathematical relationship between the alcohol index and the alcohol concentration in the blood the present invention could realize the remote sensing of drunk driving; Fatigue driving has received widespread attention worldwide, although the person knows exactly whether he is tired, the science and technology community is still perplexed about how to qualify and quantify the fatigue. However, the fatigue could be qualified and quantified by characterizing the electromagnetic dynamic changes of partial or entire human nucleon-electron clusters with the sensitive reflection waves or wavebands in the 30 μm-1000 million meters band. The enough person number could be arranged to satisfy the statistic requirement and guarantee the correctness, repeatability, reliability and comparability of the data, under the standard experiment parameters, the reflection spectra of active driver and tired driver could be extracted for comparison, the reflection spectrum of partial or entire tired driver media will show some significant changes while comparing with the active physiological state spectrum, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves, through the comparisons of reflection wave data of the active driver and the tired driver at the sensitive waves or wavebands, the reflection rate differences, the frequency differences or the phase differences at the chosen frequencies or frequency bands could be defined as the fatigue indexes to reveal the tired drivers, through establishing the qualitative and semi-quantitative relationship of the fatigue indexes and the self-evaluated fatigue degrees, the present invention could realize the remote sensing of fatigue driving, a driver's physiology state is constantly changing during driving, as time goes on, his fatigue degrees could keep getting more and more serious, the remote sensing equipment at each intersection could use the sensitive electromagnetic wave probes to obtain the fatigue indexes, if the fatigue index reach the standard limit, the loudspeaker preset in the driver's cab could be turned on for emergency stimulations, the driver could be requested to pull over to a safe area to rest so as to avoid the accident and other loss; the angry emotion caused by various factors could lead to abnormal electromagnetic changes of human head, chest or entire nucleon-electron clusters, in order to satisfy the statistic requirement and guarantee the correctness, repeatability, reliability and comparability of the data, the enough person number should be arranged to attend the relevant experiments, under the standard experiment parameters, the reflection spectra of calm person and angry person could be extracted for comparison, the reflection spectrum of the angry person electromagnetic media will show some significant changes while comparing with the calm mood state spectrum, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves of partial or entire person electromagnetic media, then the reflection rate differences, the frequency differences or the phase differences at the chosen frequencies or frequency bands could be defined as the anger indexes to reveal the angry drivers, through establishing the qualitative and semi-quantitative relationship of the anger indexes and the self-evaluated anger degrees the present invention could realize the remote sensing of road rage driving.

The same procedure could be applied in the chicken physiology monitoring, single or multiple sensitive electromagnetic frequency or frequency bands probes could be chosen from the reflection spectrum troughs, which could transform each chicken to different reflection rates or phases as its physiology indexes, the same procedure could be applied in other animal species, the different points might be the different probe frequencies or frequency bands and the different physiology index values.

The same procedure could be applied in the apple tree physiology monitoring, single or multiple sensitive electromagnetic frequency or frequency bands probes could be chosen from the reflection spectrum troughs, which could transform each apple tree to different reflection rates or phases as its physiology indexes, the rainfall amount is the crucial factor of the plant survival, if no rainfall for a long time, the apple tree could show significant changes in its reflection spectrum while comparing with the normal physiological state, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves of partial or entire apple tree electromagnetic media, the reflection rate differences, the frequency differences or the phase differences at the chosen frequencies or frequency bands could be defined as the water indexes to reveal the drought state, through establishing the quantitative relationship of the water indexes and the stem water amount the present invention could realize the remote sensing of apple tree drought levels, the apple trees with fully mature apples could show some significantly different reflection spectrum troughs while comparing with the apple trees without any apples, single or multiple wavelengths or wavebands which show the significant differences could be chosen as the electromagnetic wave probes, which could transform the apples to the apple indexes for the remote sensing of apple mature degrees; the same procedure could be applied in other plant species, the different points might be the different probe frequencies or frequency bands and the different physiology index values.

All of the reflection wave, scattering wave, absorption wave, and transmission wave come from the same emission wave, any electromagnetic changes of the reflection wave could also be manifested through the electromagnetic changes of the scattering wave, refraction wave, absorption wave, transmission wave and diffraction wave, theoretically, the reflection wave, scattering wave, absorption wave and transmission wave could be used in this invention.

The present invention could be used in the real-time remote sensing of the physiological or psychological dynamics of human, animal or plant individuals, and the relevant electromagnetic data might be useful in the areas such as drunk driving, fatigue driving, road rage driving, big data and big ecology, etc.

1. The claimed invention is a method, which title is the remote sensing of physiological dynamic electromagnetic big data, the closest current technology is the visible light photography technology, the visible light reflection wave could represent the color features of biological surface, the common feature of the present invention and the visible light photography technology is using electromagnetic wave as the detection tool.
The 0.35 μm-0.75 μm band visible light photography technology is seriously interfered by the rain-snow-fog due to the wavelength range, the plane angle and the elevation angle of visible light for extracting the psychological information of joy, anger, worry, anxiety, sadness, fear, and scare are limited, it is impossible to collect enough psychological information when the plane angle and the elevation angle are beyond certain values, limited by the wavelength range, the penetration depth of the reflection wave at the interface is less than 0.75 μm theoretically, the physiological information extracted by visible light is the surface data of usually opaque organisms, the qualitative ability of visible light is mainly featured in color discrimination, while the superficial color data could show very limited physiology value.
The reflection wave and the scattering wave could be more convenient tools while comparing the reflection, scattering, refraction, absorption, transmission and diffraction waves, the mechanisms of the reflection wave and the scattering wave are different, the reflection direction could be decided by the angle of the incident wave and the interface, according to the mechanisms of Rayleigh scattering, Raman scattering and Mie scattering the scattering directions could be distributed in the front, back or other directions, theoretically the scattering wave could be detected at the reflection wave detection point, therefore, the scattering wave at the reflection direction could be combined to the reflection wave and detected as the overall reflection wave in the actual application.
Based on the intrinsic property of the electromagnetic wave, the longer is the wavelength, the deeper is the interface penetration depth, through the exterior-interior electromagnetic interactions of vibration, rotation, polarization, magnetization or conduction at the intrinsic electromagnetic frequencies of human, animal and plant electromagnetic media, and out of the same emission wave source, any respective changes of reflection, scattering, refraction, absorption, transmission and diffraction waves could reveal electromagnetic parameter fluctuations of the physiological or psychological dynamics at the sensitive frequencies or frequency bands, which could be shown as the frequency, intensity or phase values.
The technical feature of the present invention is that the 30 μm-1000 million meters band electromagnetic waves, which include terahertz, millimeter, centimeter, decimeter, meter, radio frequency and low frequency waves, could be used to irradiate human, animal or plant individuals and extract the reflection waves to represent the nucleon-electronic clusters electromagnetic dynamic information of physiological and psychological media, such as nucleon, electron, atom, molecule, ion, water, fluid, blood, DNA, RNA, amino acid, sugar, fat, protein, cell, tissue, organ, bone, fruit, flower, leave, stem, bark, and root, etc., the nucleon-electron clusters of about one hundred kinds of elements are the entire parts of human, animal or plant, there are not other parts in organisms, any physiological or psychological states are the electromagnetic dynamic representations of nucleon-electron clusters, the three-dimensional assembly of massive nucleon-electron clusters of different properties, quantities and distributions of about one hundred kinds of elements could not only determine the differences of the electromagnetic structures of any human, animal or plant individuals, but also determine the similarity of the electromagnetic structures of the specific organism species individuals, the electromagnetic coefficients like electrical conductivity σ, relative dielectric constant εᵣ and magnetic susceptibility χₘ of various physiological media such as skin, muscle, fat, fluid, blood, vessel, organ, bone, fruit, flower, leave, stem, bark, stem and root are different from each other, the linear equations such as J_{c}=σE, D=εᵣε₀E and M=χₘH show that different electromagnetic coefficients lead to different electromagnetic energy conversion efficiencies, the 30 μm-1000 million meters band electromagnetic waves could irradiate nucleon-electron clusters of human, animal or plant individuals and cause various strong-weak electromagnetic interactions of vibration, rotation, polarization, magnetization or conduction at different frequencies, in the meantime, the electromagnetic energy conversion is related to the electromagnetic coefficients such as electrical conductivity a, relative dielectric constant εᵣ and magnetic susceptibility χₘ of physiological media, therefore, the spectrum features revealed by the interactions between the different nucleon-electron clusters of human, animal or plant individuals and the 30 μm-1000 million meters band electromagnetic waves are different from each other, in the same time, the spectrum features of the specific species are similar, human, animal or plant assembled by the astronomical nucleon-electron clusters could show not only small electromagnetic dynamic fluctuations but also significant electromagnetic dynamic changes related to alcohol, fatigue, anger and drought, etc., the physiological or psychological states of human, animal or plant individuals could be determined qualitatively and quantitatively by measuring the small or significant electromagnetic changes of their nucleon-electron clusters, the 30 μm-1000 million meters band electromagnetic waves, which wavelengths expand from micrometer to one thousand million meters, could extract the exterior-interior electromagnetic data of non-visible light properties, the reflection wave interface penetration detection could cover partial or entire electromagnetic media of human, animal or plant individuals at various depths from surface to the depth of less than one thousand million meters theoretically, the 30 μm-1000 million meters band electromagnetic waves could be used to extract the reflection spectrum of partial or entire human media in normal physiological state, the similar spectra of human individuals could show single or multiple reflection spectrum troughs, which could indicate the sensitive wavelengths or wavebands where the specific electromagnetic waves could interact with the nucleon-electron clusters at the specific frequencies or frequency bands, single or multiple sensitive wavelengths or wavebands of spectrum troughs could be chosen as the electromagnetic wave probes, which could be used to irradiate partial or entire human media and extract the reflection waves, the electromagnetic fluctuations of human nucleon-electron clusters could be transformed to single or multiple reflection rates and phases values with fluctuations, which could be the electromagnetic indexes to represent the normal physiological states of human individuals; when the alcohol concentration becomes higher and higher in the blood, the reflection spectrum of partial or entire human nucleon-electron clusters will show some significant changes while comparing with the non-drinking physiological state spectrum, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves, then the reflection rate differences, the frequency differences and the phase differences at the chosen frequencies or frequency bands could be defined as the alcohol indexes to reveal the drunk drivers, through establishing the mathematical relationship between the alcohol index and the alcohol concentration in the blood the present invention could realize the remote sensing of drunk driving; if a driver continues to drive a car for a long time, the reflection spectrum of partial or entire electromagnetic media of the tired driver will show some significant changes while comparing with the active state spectrum, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves of partial or entire electromagnetic media of the driver, then the reflection rate differences, the frequency differences or the phase differences at the chosen frequencies or frequency bands could be defined as the fatigue indexes to reveal the tired drivers, through establishing the qualitative or semi-quantitative relationship of the fatigue indexes and the self-evaluated fatigue degrees the present invention could realize the remote sensing of fatigue driving; the angry emotion caused by various factors could lead to abnormal electromagnetic changes of head, chest or entire human electromagnetic media, the reflection spectrum of electromagnetic media of the angry driver will show some significant changes while comparing with the calm mood state spectrum, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves of partial or entire driver electromagnetic media, then the reflection rate differences, the frequency differences or the phase differences at the chosen frequencies or frequency bands could be defined as the anger indexes to reveal the angry drivers, through establishing the qualitative or semi-quantitative relationship of the anger indexes and the self-evaluated anger degrees the present invention could realize the remote sensing of road rage driving; the same procedure could be applied in the chicken physiology monitoring, single or multiple sensitive electromagnetic frequencies or frequency bands probes could be chosen from the reflection spectrum troughs, which could transform each chicken to different reflection rates or phases as its physiology indexes, the same procedure could also be applied in other animal species, the different points might be the different probe frequencies or frequency bands and the different electromagnetic index values; the same procedure could be applied in the apple tree physiology monitoring, single or multiple sensitive electromagnetic frequencies or frequency bands probes could be chosen from the reflection spectrum troughs, which could transform each apple tree to different reflection rates or phases as its physiology indexes, the rainfall amount is the crucial factor of the plant survival, if no rainfall for a long time, the apple tree could show significant changes in its reflection spectrum while comparing with the normal physiological state, the reflection rates of some troughs might change significantly, the frequencies of some troughs might change significantly, some new troughs might show up, single or multiple wavelengths or wavebands which show the significant differences could be chosen as the electromagnetic wave probes to extract the reflection waves of partial or entire apple tree electromagnetic media, the reflection rate differences, the frequency differences or the phase differences at the chosen frequencies or frequency bands could be defined as the water indexes to reveal the drought state, through establishing the quantitative relationship of the water indexes and the stem water amount the present invention could realize the remote sensing of apple tree drought levels; the same procedure could be applied in other plant species, the different points might be the different probe frequencies or frequency bands and the different electromagnetic index values.
While comparing with the surface color data of visible light property, the 30 μm-1000 million meters waveband could collect the exterior-interior electromagnetic data of the nuclear-electron clusters and represent the different electromagnetic information of non-visible light property, the 30 μm-1000 million meters waveband electromagnetic waves, which longer wavelengths could show better resistance to rain-snow-fog than the visible light, could extract biological electromagnetic information for remote sensing of physiological or psychological dynamics at different observation angles, different from the visible light photography technology, the advanced technical features of the present invention, such as three-dimensional exterior-interior electromagnetic detection, physiological or psychological remote sensing, resistance to rain-snow-fog interference and no observation angle limitation could be based on the relevant electromagnetic theories, therefore, the advantages of the present invention are remarkable while comparing with the visible light photography technology.
The 30 μm-1000 million meters electromagnetic waveband is commonly used in the fields of frequency modulation, amplitude modulation, television and communication, the electromagnetic wave and nucleon-electron clusters are well known, so far there is no patent or non-patent literature which invents that single or multiple sensitive wavelengths or wavebands of the 30 μm-1000 million meters waveband spectra could be used as the electromagnetic wave probes in the remote sensing of various physiological or psychological states of human, animal or plant individuals, such as drunk driving, fatigue driving, road rage driving and soil drought, etc.
The technical scope claimed by the present invention is a method of remote sensing of the physiology or psychology electromagnetic dynamics, the 30 μm-1000 million meters waveband electromagnetic waves could be used to scan the partial or entire nucleon-electron clusters of human individuals in the normal physiological or psychological states to extract the respective spectra of reflection wave, scattering wave, absorption wave or transmission wave, single or multiple wavelengths or wavebands of spectrum troughs or peaks could be chosen as the electromagnetic wave probes, which could be used to irradiate partial or entire nucleon-electron clusters of human individuals, extract the respective electromagnetic wave and transform the human individuals to respective electromagnetic indexes through the electromagnetic parameters values at the specific frequencies or frequency bands, such as the frequency, reflection rate, scattering rate, absorption, transmission rate or phase, etc., the electromagnetic dynamics of different physiology or psychology states of human individuals like sleep, exercise, alcohol, fatigue or anger could be revealed through the significant changes in the electromagnetic spectra, while comparing with the normal physiological or psychological states, the parameter values of some troughs or peaks might change significantly, the frequencies of some troughs or peaks might change significantly, some new troughs or peaks might show up, single or multiple wavelengths or wavebands which show the significant differences could be chosen as the electromagnetic wave probes to extract the electromagnetic parameter values of partial or entire human electromagnetic media, the reflection rate differences, the scattering rate differences, absorption differences, transmission rate differences, the frequency differences or the phase differences at the chosen frequencies or frequency bands could be defined as the electromagnetic indexes to represent the electromagnetic dynamics of the human individuals in different physiology or psychology states, through the comparisons of the electromagnetic indexes in the normal physiology or psychology states and other physiology or psychology states, this invention could realize the remote sensing of drunk driving, fatigue driving, road rage driving, big data and big ecology, etc., the same method could also be used in each animal or plant species.
